(19) United States
(12) Patent Application Publication
(10) Pub. No.: US 2024/0149171 A1
(43) Pub. Date: May 9, 2024

(54) SYSTEM AND METHOD FOR CREATING A CRYPTOGRAPHICALLY SECURED DIGITAL ASSET

(71) Applicant: Orpyx Medical Technologies Inc., Calgary (CA)

(72) Inventors: Cory David PADDOCK, CALGARY (CA); Julia Breanne EVERETT, CALGARY (CA); Joanna Klara MAJCHERKIEWICZ, CALGARY (CA); Samuel Carl William BLADES, VICTORIA (CA); Tyler Alexander FRASER, CALGARY (CA); Jeffrey Thompson KNIGHT, AUSTIN, TX (US)

(21) Appl. No.: 18/549,684

(22) PCT Filed: Mar. 14, 2022
(86) PCT No.: PCT/CA2022/050371

§ 371 Date: Sep. 8, 2023

Related U.S. Application Data
(60) Provisional application No. 63/179,734, filed on Apr. 26, 2021.
(60) Provisional application No. 63/163,719, filed on Mar. 19, 2021.

(51) Int. Cl. A63F 13/73; A61B 5/00; A61B 5/11; A61B 5/117; A63F 13/211; A63F 13/212; A63F 13/213; A63F 13/217; A63F 13/218; G06F 21/60; G06F 21/62; G16H 40/67

(52) U.S. Cl. CPC A63F 13/73; A61B 5/1118; A61B 5/117; A61B 5/6807; G06F 21/602; G06F 21/6245; G16H 40/67; A61B 2503/10; A63F 13/211; A63F 13/212; A63F 13/213; A63F 13/217; A63F 13/218

(57) ABSTRACT

A method for creating a cryptographically secured digital asset includes: a. using at least a first activity data sensor to collect data relating to an activity carried out by a user, b. transmitting the data to a processing system, c. using the processing system to generate a digital asset based on the data, and d. cryptographically securing the digital asset. A system for creating a cryptographically secured digital asset includes at least a first activity data sensor configured to collect data relating to an activity carried out by a user, and a processing system configured to receive the data from the first activity data sensor, generate a digital asset based on the data, and cryptographically secure the digital asset.

### CROSS-REFERENCES TO RELATED APPLICATIONS

This application claims the benefit of and/or priority to U.S. Provisional Patent Application No. 63/163,719 filed on Mar. 19, 2021, and U.S. Provisional Patent Application No. 63/179,734 filed on Apr. 26, 2021, both of which are incorporated herein by reference in their entirety.

### FIELD

This document relates to cryptographically secured digital assets. More specifically, this document relates to systems and methods for creating a cryptographically secured digital asset, such as a digital asset that is cryptographically secured using blockchain technology.

### BACKGROUND

U.S. Pat. No. 10,505,726 (to Andon et al.) discloses cryptographic digital assets for articles of footwear, methods for making/using such cryptographic digital assets, and decentralized computing systems with blockchain control logic for mining, intermingling, and exchanging blockchain-enabled digital shoes. A method of generating cryptographic digital assets for footwear includes a middleware server computer receiving, over a distributed computing network from a remote computing node, confirmation of a validated transfer of footwear from one party to another. The middleware server computer determines, from an encrypted relational database, a unique owner ID code of the transferee party, and generates a cryptographic digital asset for the footwear. This cryptographic digital asset includes a digital shoe and a unique digital shoe ID code. The middleware server computer links the cryptographic digital asset with the unique owner ID code, and records the unique digital shoe ID code and the unique owner ID code on a transaction block with a distributed blockchain ledger.

U.S. Patent Application Publication No. 2019/0363874 (to Shirley et al.) discloses systems, methods, and media for providing secure and scalable decentralized computation for one or more decentralized applications. Features include a blockchain; and a plurality of nodes comprising a plurality of each of three specialized node types, the specialized node types comprising access nodes, security nodes, and execution nodes.

### SUMMARY

The following summary is intended to introduce the reader to various aspects of the detailed description, but not to define or delimit any invention.

Methods for creating cryptographically secured digital assets are disclosed. According to some aspects, a method for creating a cryptographically secured digital asset includes: a. using at least a first activity data sensor to collect data relating to an activity carried out by a user, b. transmitting the data to a processing system, c. using the processing system to generate a digital asset based on the data, and d. cryptographically securing the digital asset.

In some examples, the first activity data sensor is a somatic sensor. The somatic sensor can be a force sensor, a shear sensor, a weight sensor, a body-mass-index sensor, a temperature sensor, a moisture sensor, a heart rate sensor, a heart rate variability sensor, a blood pressure sensor, a blood flow sensor, a cardiac output sensor, a perfusion sensor, an inductance sensor, a resistance sensor, a dielectric sensor, a capacitance sensor, a conductance sensor, an odor sensor, a taste sensor, a hydration sensor, a respiratory flow rate sensor, an accelerometer, a gyroscope, a magnetometer, a limb positioning measurement device, a sensor for detecting the presence of a chemical marker, a blood glucose sensor, a sweat sensor, a blood alcohol sensor, an oxygen sensor, a carbon dioxide sensor, a drug level sensor, an electrolyte sensor, a pH sensor, an acidity sensor, an EEG sensor, an EMG sensor, an ECG sensor, a lung function meter, an impairment sensor, a sleep sensor, a body fat sensor, a height sensor, a fatigue sensor, an audio sensor, an RF sensor, an airflow meter, a spirometer, a lung volume sensor, a facial recognition sensor, a voice sensor, an emotion sensor, a stress sensor, an endorphin sensor, a cortisol sensor, an adrenaline sensor, an infrared sensor, or a bacterial load sensor. In one particular example, the somatic sensor is a force sensor.

In some examples, the first activity data sensor is a non-somatic sensor. The non-somatic sensor can be a timer, a weather sensor, a GPS sensor, an audio sensor, a light sensor, an altimeter, radar, lidar, a milestone sensor, an equipment-based sensor, or a proximity sensor.

In some examples, step a. includes collecting the data while the first activity data sensor is carried by a wearable device worn by the user. For example, step a. can include collecting the data while the first activity data sensor is carried by an insole worn by the user.

In some examples, the activity is or includes a gaming activity.

In some examples, the activity is or includes an athletic activity.

In some examples, the method further includes using the data to authenticate at least one statistic relating to the activity.

The activity can include at least one of running, jogging, walking, swimming, skiing, show-shoeing, high-jump, long-jump, pole-vault, biathlon, heptathlon, decathlon, pentathlon, skateboarding, cycling, figure skating, speed skating, basketball, baseball, soccer, hockey, lacrosse, artistic swimming, archery, badminton, softball, volleyball, boxing, martial arts, dancing, canoeing, diving, equestrian, fencing, football, golf, gymnastics, handball, rowing, rugby, sailing, shooting, climbing, surfing, table tennis, tennis, trampoline, triathlon, water polo, weightlifting, wrestling, fighting, disc throwing, balancing, bobsleigh, curling, luge, skeleton, snowboarding, roller skating, cross-fit, dance, a Paralympic sport, and an adapted sport.

In some examples, the digital asset includes a visual representation of the data. Step c. can include applying a set of instructions to the data to generate the visual representation of the data. Step c. can include applying a set of instructions to the data to generate an abstract visual representation of the data. Step c. can include applying a set of instructions to the data to generate a figurative visual representation of the data.

In some examples, the digital asset includes an audio representation of the data. Step c. can include applying a set of instructions to the data to generate the audio representation of the data.

In some examples, the digital asset is based on only the data. In some examples, the digital asset is based on the data and additional non-sensor based data relating to the activity carried out by the user. The additional non-sensor based data can include at least one of weather data, historical data, game statistic data, count data, score data, race data, GPS data, audio data, photographic data, videographic data, known body measurement data, data from one or more additional users, data regarding surroundings, ground material data, apparel data, activity type data, equipment-based data, survey data, feedback data, personal information, and social surroundings data.

In some examples, the method includes using the data to authenticate the user. The first activity data sensor can be a force sensor positioned on a sole of the user's foot, the data can include force applied between the user's foot and a surface, and using the data to authenticate the user can include using the processing system to analyze the user's gait based on the data, and authenticating the user based on the analysis of the user's gait.

In some examples, the method further includes using the processing system to automatically detect a nature of the activity.

In some examples, the method includes using the processing system to analyze the data. The digital asset can be based on the analysis of the data.

In some examples, the digital asset is generated proportionally to the data.

In some examples, the cryptographically secured digital asset is or includes a skin.

In some examples, the cryptographically secured digital asset includes an attribute that is usable in gaming.

In some examples, the method further includes using the cryptographically secure digital asset in gaming.

In some examples, the method further includes transferring ownership of the cryptographically secured digital asset to a third party.

Systems for creating cryptographically secured digital assets are also disclosed. According to some aspects, a system for creating a cryptographically secured digital asset includes at least a first activity data sensor configured to collect data relating to an activity carried out by a user, and a processing system configured to receive the data from the first activity data sensor, generate a digital asset based on the data, and cryptographically secure the digital asset.

In some examples, the first activity data sensor is a somatic sensor. The somatic sensor can be a force sensor, a shear sensor, a weight sensor, a body-mass-index sensor, a temperature sensor, a moisture sensors, a heart rate sensor, a heart rate variability sensor, a blood pressure sensor, a blood flow sensor, a cardiac output sensor, a perfusion sensor, an inductance sensor, a resistance sensor, a dielectric sensor, a capacitance sensor, a conductance sensor, an odor sensor, a taste sensor, a hydration sensor, a respiratory flow rate sensor, an accelerometer, a gyroscope, a magnetometer, a limb positioning measurement device, a sensor for detecting the presence of a chemical marker, a blood glucose sensor, a sweat glucose sensor, a blood alcohol sensor, an oxygen sensor, a carbon dioxide sensor, a drug level sensor, an electrolyte sensor, a pH sensor, an acidity sensor, an EEG sensor, an EMG sensor, an ECG sensor, a lung function meter, an impairment sensor, a sleep sensor, a body fat sensor, a height sensor, a fatigue sensor, an audio sensor, an RF sensor, an airflow meter, a spirometer, a lung volume sensor, a facial recognition sensor, a voice sensor, an emotion sensor, a stress sensor, an endorphin sensor, a cortisol sensor, an adrenaline sensor, an infrared sensor, or a bacterial load sensor.

In some examples, the first activity data sensor is a non-somatic sensor. The non-somatic sensor can be a timer, a weather sensor, a GPS sensor, an audio sensor, a light sensor, an altimeter, radar, lidar, a milestone sensor, an equipment-based sensor, or a proximity sensor.

In some examples the system further includes a wearable device that carries the first activity data sensor. For example, the system can further include an insole that carries the first activity data sensor. In some examples, the system further includes a piece of gaming equipment that carries the first activity data sensor.

In some examples, the digital asset includes a visual representation of the data. In some examples, the digital asset includes an abstract visual representation of the data. In some examples, the digital asset includes a figurative visual representation of the data.

In some examples, the processing system is configured to apply a set of instructions to the data to generate the digital asset.

In some examples, the processing system is configured to generate the digital asset based on only the data.

In some examples, the processing system is configured to generate the digital asset based on the data and additional non-sensor based data relating to the activity carried out by the user. The additional non-sensor based data can include at least one of weather data, historical data, game statistic data, count data, score data, race data, GPS data, audio data, photographic data, videographic data, known body measurement data, biometric data from one or more additional users, data regarding surroundings, ground material data, apparel data, activity type data, equipment-based data, survey data, feedback data, personal information, and social surroundings data.

In some examples, the processing system is configured to use the data to authenticate the user. The first activity data can be a force sensor that is positionable on a sole of the user's foot and that is configured to measure force applied between the user's foot and a surface, and the processing device can be configured to analyze the user's gait based on the data, and authenticate the user based on the analysis of the user's gait.

In some examples, the processing system is configured to use the data to authenticate at least one statistic relating to the activity.

In some examples, the processing system is configured to automatically detect a nature of the activity.

In some examples, processing system is further configured to analyze the data, and the digital asset is based on the analysis of the data.

In some examples, the system is configured to generate the digital asset proportionally to the data.

In some examples, the cryptographically secured digital asset includes an attribute that is usable in gaming.

In some examples, the cryptographically secured digital asset is a skin or includes a skin.

According to some aspects, a method for creating a cryptographically secured digital asset includes: a. collecting data relating to a gaming activity carried out by a user; b. transmitting the data to a processing system; c. using the processing system to generate a digital asset based on the data; and d. cryptographically securing the digital asset.

In some examples, step a. includes collecting the data with a first activity data sensor.

In some examples, the first activity data sensor is a somatic sensor. The somatic sensor can be a force sensor, a shear sensor, a weight sensor, a body-mass-index sensor, a temperature sensor, a moisture sensor, a heart rate sensor, a heart rate variability sensor, a blood pressure sensor, a blood flow sensor, a cardiac output sensor, a perfusion sensor, an inductance sensor, a resistance sensor, a dielectric sensor, a capacitance sensor, a conductance sensor, an odor sensor, a taste sensor, a hydration sensor, a respiratory flow rate sensor, an accelerometer, a gyroscope, a magnetometer, a limb positioning measurement device, a sensor for detecting the presence of a chemical marker, a blood glucose sensor, a sweat glucose sensor, a blood alcohol sensor, an oxygen sensor, a carbon dioxide sensor, a drug level sensor, an electrolyte sensor, a pH sensor, an acidity sensor, an EEG sensor, an EMG sensor, an ECG sensor, a lung function meter, an impairment sensor, a sleep sensor, a body fat sensor, a height sensor, a fatigue sensor, an audio sensor, an RF sensor, an airflow meter, a spirometer, a lung volume sensor, a facial recognition sensor, a voice sensor, an emotion sensor, a stress sensor, an endorphin sensor, a cortisol sensor, an adrenaline sensor, an infrared sensor, or a bacterial load sensor.

In some examples, the first activity data sensor is a non-somatic sensor. The non-somatic sensor can be a timer, weather sensor, a GPS sensor, an audio sensor, a light sensor, an altimeter, radar, lidar, a milestone sensor, an equipment-based sensor, or a proximity sensor.

In some examples, the data includes non-sensor based data relating to the gaming activity. The non-sensor based data can include at least one of weather data, historical data, game statistic data, count data, score data, race data, GPS data, audio data, photographic data, videographic data, known body measurement data, data from one or more additional users, data regarding surroundings, ground material data, apparel data, activity type data, equipment-based data, survey data, feedback data, personal information, and social surroundings data.

In some examples, step a. includes collecting the data with at least a first activity data sensor that is carried by a piece of gaming equipment.

In some examples, the method further includes using the data to authenticate at least one statistic relating to the gaming activity.

In some examples, the method further includes using the data to authenticate the user.

In some examples, the method further includes using the processing system to analyze the data, and the digital asset can be based on the analysis of the data.

In some examples, the digital asset is generated proportionally to the data.

In some examples, the digital asset includes a skin.

In some examples, the cryptographically secured digital asset includes an attribute that is usable in the gaming activity.

In some examples, the method further includes using the cryptographically secured digital asset in the gaming activity.

In some examples, the method further includes transferring ownership of the cryptographically secured digital asset to a third party.

In some examples, the data is collected by a camera that is carried by a piece of gaming equipment.

### BRIEF DESCRIPTION OF THE DRAWINGS

The drawings included herewith are for illustrating various examples of articles, methods, and apparatuses of the present specification and are not intended to limit the scope of what is taught in any way. In the drawings:

FIG. 1 is a schematic view of an example system for creating a cryptographically secured digital asset;

FIG. 2 is a flowchart of an example method for creating a cryptographically secured digital asset;

FIG. 3 shows an example user interface of an app that can generate a digital asset based on data collected by an activity data sensor; and

FIG. 4 shows another example user interface of an app that can generate a digital asset based on data collected by an activity data sensor.

### DETAILED DESCRIPTION

Various apparatuses or processes or compositions will be described below to provide an example of an embodiment of the claimed subject matter. No embodiment described below limits any claim and any claim may cover processes or apparatuses or compositions that differ from those described below. The claims are not limited to apparatuses or processes or compositions having all of the features of any one apparatus or process or composition described below or to features common to multiple or all of the apparatuses or processes or compositions described below. It is possible that an apparatus or process or composition described below is not an embodiment of any exclusive right granted by issuance of this patent application. Any subject matter described below and for which an exclusive right is not granted by issuance of this patent application may be the subject matter of another protective instrument, for example, a continuing patent application, and the applicants, inventors or owners do not intend to abandon, disclaim or dedicate to the public any such subject matter by its disclosure in this document.

For simplicity and clarity of illustration, reference numerals may be repeated among the figures to indicate corresponding or analogous elements. In addition, numerous specific details are set forth in order to provide a thorough understanding of the subject matter described herein. However, it will be understood by those of ordinary skill in the art that the subject matter described herein may be practiced without these specific details. In other instances, well-known methods, procedures and components have not been described in detail so as not to obscure the subject matter described herein. The description is not to be considered as limiting the scope of the subject matter described herein.

The terms “coupled” or “coupling” as used herein can have several different meanings depending in the context in which these terms are used. For example, the terms coupled or coupling can have a mechanical, electrical or communicative connotation. For example, as used herein, the terms coupled or coupling can indicate that two elements or devices are directly connected to one another or connected to one another through one or more intermediate elements or devices via an electrical element, electrical signal, or a mechanical element depending on the particular context. Furthermore, the term “communicative coupling” may be used to indicate that an element or device can electrically, optically, or wirelessly send data to another element or device as well as receive data from another element or device.

As used herein, the wording “and/or” is intended to represent an inclusive-or. That is, “X and/or Y” is intended to mean X or Y or both, for example. As a further example, “X, Y, and/or Z” is intended to mean X or Y or Z or any combination thereof. Furthermore, the wording “at least one of X and Y” is intended to mean only X, only Y, or both X and Y.

Terms of degree such as “substantially”, “about” and “approximately” as used herein mean a reasonable amount of deviation of the modified term such that the end result is not significantly changed. These terms of degree may also be construed as including a deviation of the modified term if this deviation would not negate the meaning of the term it modifies.

Any recitation of numerical ranges by endpoints herein includes all numbers and fractions subsumed within that range (e.g. 1 to 5 includes 1, 1.5, 2, 2.75, 3, 3.90, 4, and 5). It is also to be understood that all numbers and fractions thereof are presumed to be modified by the term “about” which means a variation of up to a certain amount of the number to which reference is being made if the end result is not significantly changed.

As used herein the term “gaming” refers to the use of video games—i.e. an electronic game that involves interaction with a user interface or input (such as a controller, keyboard, joystick, headset, goggles or motion sensing device). Similarly, the term “gamer” refers to a user of a video game. The term “skin” refers to a graphic or audio file that changes the appearance and/or sound of an element in a video game (e.g. a skin can be a shirt worn by an avatar), or that adds an element in a video game (e.g. a skin can be a tool used by an avatar).

Generally disclosed herein is a method for creating a cryptographically secured digital asset, and related systems. The methods can generally involve collecting data using one or more activity data sensors, using the data to create a digital asset, and cryptographically securing the digital asset (e.g. with a fungible or non-fungible token, such as a non-fungible token registered on and validated by a blockchain platform). The data can relate to an activity carried out by a user (e.g. an athletic activity or a gaming activity), and the digital asset can be based on the data, so that the digital asset can represent the activity (e.g. the digital asset can be a piece of digital art that is created by applying a set of instructions such as an algorithm to the data, or can be a skin that is earned based on performance). More specifically, the methods can generally include using at least a first activity data sensor to collect data relating to an activity carried out by a user, transmitting the data to a processing system, using the processing system to generate a digital asset based on the data, and cryptographically securing the digital asset. The systems can generally include at least a first activity data sensor configured to collect data relating to an activity carried out by a user, and a processing system configured to receive the data from the first activity data sensor, generate a digital asset based on the data, and cryptographically secure the digital asset.

The systems and methods described herein can in some examples allow for elite performances—e.g. elite athletic or gaming performances—to be memorialized and optionally monetized. For example, an elite or high performance athlete or gamer can perform an activity, and a cryptographically secured digital asset based on data collected during that activity can be created. That cryptographically secured digital asset can then become a collector's item. In a more specific example, an elite sprinter can use an activity data sensor to collect data during a high-profile event (e.g. an Olympic final). A digital asset can then be created based on the data. For example, a set of instructions can be applied to the data to create a digital asset in the form of a piece of abstract art. The digital asset can then be cryptographically secured, and optionally sold (e.g. sold peer to peer or on a commerce platform), traded, gifted, or collected.

In one specific non-limiting example that will be described in further detail below, a user can wear an insole that includes one or more force sensors that sense the force applied by the user's foot to a surface. Such insoles are described, for example, in U.S. Pat. No. 10,004,428 (Everett et al.) and United States Patent Application Publication No. 2020/0218974 (Cheng et al.), both of which are incorporated herein by reference in their entirety. The user can then engage in an activity, such as a run or other athletic activity, or a gaming activity (e.g. a movement-based video game, such as a dance-based video game). During the activity, the sensors in the insole can collect data. During or after the activity, the data can be transmitted to a processing system (e.g. wirelessly, or by physically connecting the insole to the processing system), and the processing system can generate a digital asset based on the data (either autonomously or with the input of the user or a third party), such as an image that contains a representation of the data (e.g. a graph showing the data or a piece of abstract art that represents the data). The digital asset can then be cryptographically secured.

As used herein, the term “activity data sensor” can refer to any sensor that detects or measures a physical property relating to an activity carried out by a user, to collect data relating to the activity. Activity data sensors can include “somatic sensors” and “non-somatic sensors”.

As used herein, the term “somatic sensor” refers to an activity data sensor that detects or measures a physical property of a user's body. General examples of somatic sensors include physiologic sensors, biometric sensors, anatomical sensors, biomechanical sensors, body position sensors, and/or biochemical sensors (e.g. for sensing a property of blood, sweat, and/or urine). Specific examples of somatic sensors include force sensors (where the term “force sensor” can refer to an activity data sensor that detects or measures raw force, or an activity data sensor that measures or detects a parameter that is a function of force, such as pressure), shear sensors, weight sensors, body-mass-index sensors, temperature sensors, moisture sensors, heart rate sensors, heart rate variability sensors, blood pressure sensors, blood flow sensors, cardiac output sensors, perfusion sensors, inductance sensors, resistance sensors, dielectric sensors, capacitance sensors, conductance sensors, odor sensors, taste sensors, hydration sensors, respiratory flow rate sensors, accelerometers, gyroscopes, magnetometers, limb positioning measurement devices, sensor for detecting the presence of a chemical marker, blood glucose sensors, sweat sensors (e.g. sweat glucose sensors), blood alcohol sensors, oxygen sensors (e.g. exhaled oxygen sensors or oxygen saturation sensors), exhaled carbon dioxide sensors, drug level sensors, electrolyte sensors, pH sensors, acidity sensors, EEG sensors, EMG sensors, ECG sensors, lung function meters, impairment sensors, sleep sensors, body fat sensors, height sensors, fatigue sensors, audio sensors, RF sensors (e.g. RFID tag sensors), airflow meters, spirometers, lung volume sensors, facial recognition sensors, voice sensors, emotion sensors, stress sensors, endorphin sensors, cortisol sensors, adrenaline sensors, an infrared sensor, or bacterial load sensors.

As used herein, the term “non-somatic sensor” refers to an activity data sensor that measures a physical property that is not a physical property of a user's body. For example, non-somatic sensors can measure a physical property of the surroundings of a user. Specific non-limiting examples of non-somatic sensors include a timer, a weather sensor, a GPS sensor, an audio sensor, a light sensor, an altimeter, radar, lidar, a milestone sensor (e.g. a laser sensor at a start line or finish line), a sensor in a piece of equipment (e.g. in a ball, a puck, a net, or a game floor) or a proximity sensor.

Activity data sensors can optionally be carried by one or more wearable devices. Non-limiting examples of wearable devices that can carry activity data sensors include: insoles (as described above), sock liners, socks, orthotics, shoes, helmets, chest straps, wristbands (e.g. a smart watch), a head band, armbands, rings, necklaces, clothing, contact lenses, goggles, and/or glasses. However, it is possible that an activity data sensor is not carried by a wearable device. For example, a sensor can be adhered or affixed to a user's body, implanted in a user's body, or can be placed proximate a user's body. For further example, an activity data sensor can be carried by a piece of gaming equipment (e.g. a video game console, controller, headset or goggles). For further example, an activity data sensor can be built into an object that is carried by hand (e.g. a video game controller).

As used herein, the term “activity” can refer to any activity that involves the human body, whether moving or stationary. Non-limiting examples of activities include athletic activities, recreational activities, performances, gaming activities and everyday activities (e.g. cooking or cleaning). Example athletic activities include: running/jogging, walking, swimming, skiing (e.g. alpine, cross-country, jumping, or freestyle skiing), snow-shoeing, high-jump, long-jump, pole-vault, biathlon, heptathlon, decathlon, pentathlon, skateboarding, cycling (e.g. road biking, mountain biking, spin cycling, BMX cycling, track cycling), figure skating, speed skating, basketball, baseball, soccer, hockey, lacrosse, artistic swimming, archery, badminton, softball, volleyball, boxing, martial arts (e.g. judo, karate, taekwondo), dancing, canoeing, diving, equestrian (e.g. dressage, eventing, jumping), fencing, football, golf, gymnastics (e.g. artistic or rhythmic gymnastics), handball, rowing, rugby, sailing, shooting, climbing, surfing, table tennis, tennis, trampoline, triathlon, water polo, weightlifting, wrestling (e.g. freestyle or Greco-Roman), fighting, disc throwing, balancing, bobsleigh, curling, luge, skeleton, snowboarding, roller skating, cross-fit, dance, virtual versions of such activities, and/or Paralympic or adapted sports. Example gaming activities include the use of a video game console, an arcade game, a computer game, a remote cloud game, or a virtual or augmented reality game. The term “activity” can in some instances refer a combination of multiple activity types. For example, an activity can be both an athletic activity and a gaming activity (e.g. a running or cycling physical trainer that allows users to interact, train, and compete in a virtual world).

As used herein, the term “digital asset” can refer to any asset that exists in digital format. Non-limiting examples of digital assets include digital images, spreadsheets, text files, slide decks, digital videos, audio files, CAD files, 3D model files, skins, and combinations thereof. Digital images can include, for example, one or more emojis, one or more symbols, figurative art, abstract art, graphs, charts, plots, tables, logos, slogans, word art, holograms, and combinations thereof. Digital assets can be accompanied by metadata, such as dates, the name of the user(s) that created the digital asset, and/or the weather when the digital asset was created. Digital assets can include attributes to be used in gaming. Digital assets can be incorporated into an augmented or virtual reality experience, or into a game (e.g. as a skin in a video game) or puzzle.

As used herein, the term “processing system” can refer to a single computing device, or a plurality of computing devices that are in communication (e.g. via wired or wireless communication). For example, the term “processing system” can refer to a smart phone, a smart watch, a tablet, a computer (e.g. a laptop or desktop computer), a network of computers, a processing device built into a wearable device, a processing device built into a gaming console, a standalone processing device, or a combination thereof. For further example, the term “processing system” can refer to a combination of a user's own device (e.g. a smart phone) and a third-party platform (e.g. decentralized computing network).

As used herein, the term “cryptographically secured digital asset” refers to a digital asset that is fungible or non-fungible. Digital assets can be cryptographically secured, for example, using blockchain technology. For example, digital assets can be cryptographically secured with a fungible or non-fungible token registered on and validated by a blockchain platform (which can also be referred to as “minting a fungible or non-fungible token”), or otherwise registered in an immutable database. Such platforms include, for example, Opensea, Mintable, and Rarible.

Used herein, the phrase “set of instructions” can include (but is not limited to) algorithms such as digital image processing algorithms (e.g. filtering, smoothing, sharpening, enhancement, compression, encoding, etc.), evolutionary algorithms (e.g. genetic algorithms, metaheuristic algorithms), machine learning, recursive algorithms, fractal creation, segmentation, pattern-based algorithms, tiling, puzzle creating, pixel size alteration, abstraction, realism, layering, addition of text, addition of sound, symmetry operations, overlap operations, morphing, amplification, shading, randomization, categorization, proportion alterations, mirroring, sequencing, orientation, segmentation, creating of static and dynamic portions, and reversing.

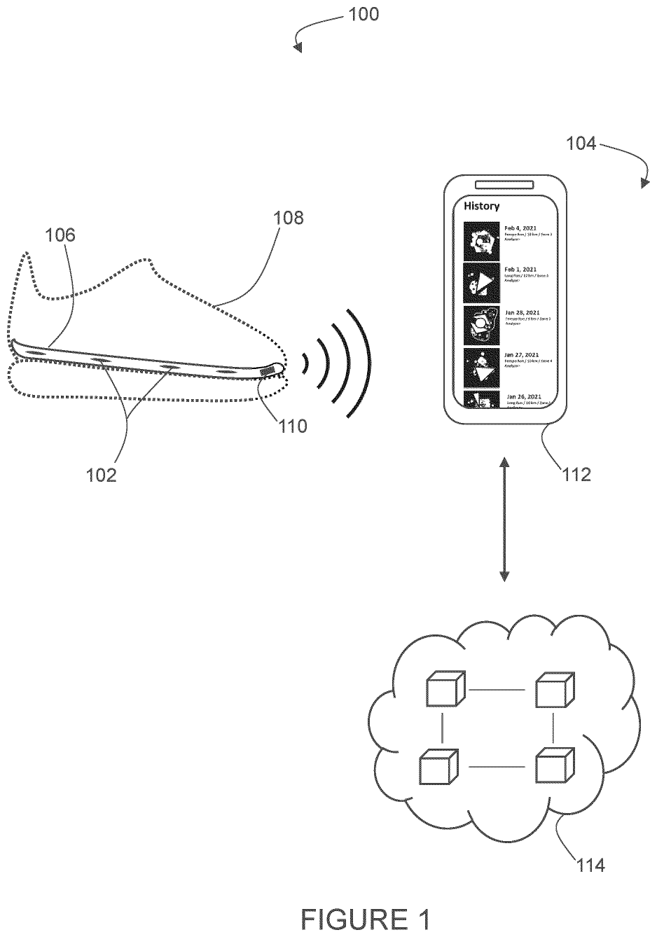

Referring now to FIG. 1, a first example of a system 100 for creating a cryptographically secured digital asset is shown. The system generally includes a set of activity data sensors 102 (i.e. a first activity data sensor, a second activity data sensor, and so on), and a processing system 104.

In the example shown, the system 100 includes a wearable device in the form of an insole 106 that is worn in a shoe 108, and the insole 106 carries the set of activity data sensors 102. The activity data sensors 102 can collect data relating to an activity carried out by a user. In the example shown, the activity data sensors 102 are in the form of force sensors, which measure the force applied by the user's foot to a surface (e.g. the ground, a treadmill belt, or a bicycle pedal). The insole 106 further includes a transmitter 110, for receiving data from the activity data sensors 102 and transmitting the data to the processing system 104. In the example shown, the transmitter 110 wirelessly transmits the data to the processing system; however, in alternative examples, the transmitter can be wired to the processing system. As noted above, insoles that include force sensors are described in detail in U.S. Pat. No. 10,004,428 (Everett et al.) and United States Patent Application Publication No. 2020/0218974 (Cheng et al.), and for simplicity, such insoles are not described in detail herein.

As mentioned above, in alternative examples, activity data sensors can be carried by another type of wearable device, or can be provided without a wearable device. Furthermore, activity data sensors can be another type of sensor, including other somatic sensors and non-somatic sensors.

Referring still to FIG. 1, the processing system 104 is configured to receive the data from the set of activity data sensors 102, generate a digital asset based on the data, and cryptographically secure the digital asset. More specifically, in the example shown, the processing system 104 includes a smartphone 112 (e.g. the user's own smartphone) and a blockchain enabled platform (e.g. a third party platform), generally indicated at 114. The smartphone 112 can wirelessly receive the data from the set of activity data sensors 102 via the transmitter 110, and in the example shown carries an app for generating a digital asset based on the data (described in further detail below). The smartphone 112 is in communication with the blockchain enabled platform 114, which can cryptographically secure the digital asset.

Optionally, the system 100 can carry secondary activity data sensors, such as one or more non-somatic sensors as described above, for collecting secondary activity data. In one specific example, the smartphone can carry a secondary activity data sensor in the form of a GPS sensor, for mapping the activity. In another specific example, the insole 106 can carry a secondary activity data sensor in the form of a GPS sensor, for mapping the activity.

In alternative examples, a processing system can be of another configuration. For example, one or more parts of a processing system can be packaged with the activity data sensors (e.g. an insole can include a processing device). For further example, a single computing device can receive the data from the set of activity data sensors, generate a digital asset based on the data, and cryptographically secure the digital asset.

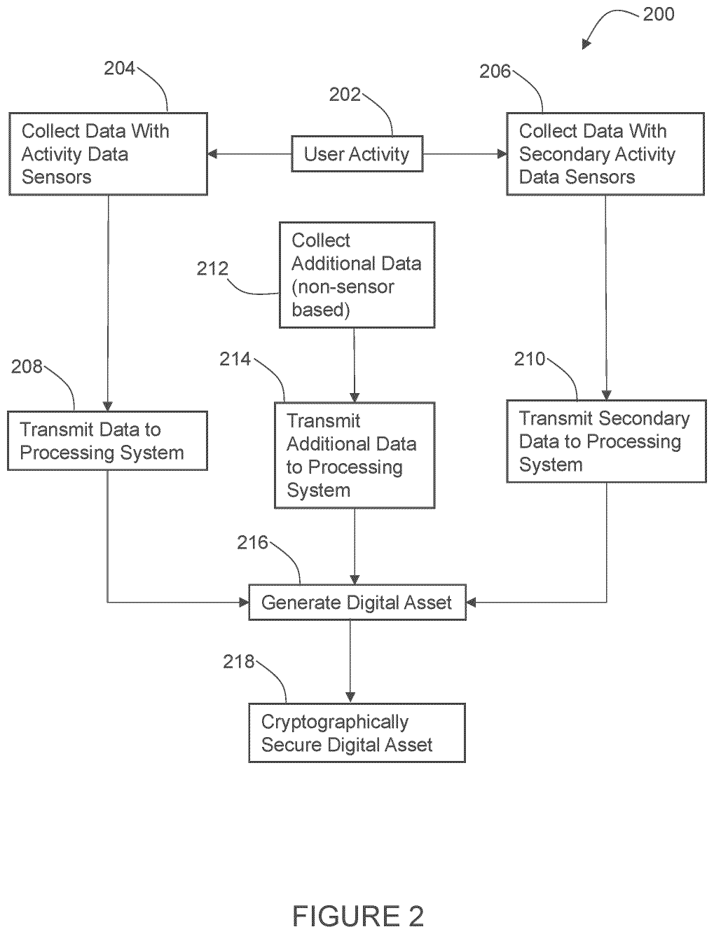

An example method 200 for creating a cryptographically secured digital asset will now be described with reference to FIG. 2. The method 200 will be described with reference to the system 100; however, the method 200 is not limited to the system 100, and the system 100 is not limited to the method 200. As will be described in further detail below, the method 200 can generally include using at least a first activity data sensor (e.g. activity data sensors 102) to collect data relating to an activity carried out by a user, transmitting the data to a processing system (e.g. processing system 104), using the processing system to generate a digital asset based on the data, and cryptographically securing the digital asset.

Referring now to FIG. 2, at the start of the method 200, the activity data sensors 102 can be used to collect data relating to an activity carried out by a user. For example, with the insole 106 positioned in a user's shoe 108 and with the activity data sensors 102 activated to collect data, the user can wear the shoe 108 and carry out an activity (step 202), for example an athletic activity such as a run or a gaming activity (or any other type of activity, as described above). During the activity, the activity data sensors 102 can collect data (step 204), for example continuously, semi-continuously, intermittently, or sporadically. Furthermore, during the activity, the secondary activity data sensors can collect secondary activity data (step 206), such as GPS data.

The data collected by the activity data sensors 102 and the secondary activity data collected can be transmitted to the processing system 104 (steps 208 and 210, respectively). For example, in real time during the activity, the data and the secondary data can be wirelessly transmitted to the processing system 104 by the transmitter 110. For further example, during the activity, the activity data sensors 102 can continuously collect data, and the transmitter 110 can continuously transmit the data to the user's smart phone 112. Alternatively, upon completion of the activity, the data can be transmitted to the smartphone 112.

Optionally, additional data relating to the activity can also be collected (step 212), where the additional data is not collected by an activity data sensor, but is otherwise known to or available to the user. Such additional data can be referred to herein as non-sensor based data, and can include, for example, weather data (e.g. precipitation, water temperature, air temperature, wind resistance, wind direction, wind speed, air quality, and/or air pressure), historical data (e.g. data from a prior activity), game statistic data (e.g. quantity of rebounds, goals, points, and/or yards), count data (e.g. number of cleans, medicine ball throws, push ups, sit ups, and/or squats), score or race data (e.g. win or loss, finishing place, and/or segment results), GPS data (where the GPS data is, for example, publicly available data and is not specifically collected in relation to the activity carried out by the user), audio data, photographic data, videographic data (e.g. videographic data collected by a video camera that is part of a piece of gaming equipment such as a gaming console), known body measurement data (e.g. height), data from one or more additional users (e.g. a training partner, opponent, or team member(s)), data regarding surroundings (e.g. whether the activity was done indoors or outdoors), ground material data (e.g. gravel, turf, laminate, grass, concrete, clay, dirt, snow, treadmill, etc.), apparel data (e.g. type of clothing or footwear), activity type data (e.g. whether the activity was a race or training), equipment-based data, social surroundings data (e.g. identity of a training partner or competitor), score or performance data from a gaming activity (e.g. a high score), personal information (e.g. the user's initials or another personal attribute), skill-level data (e.g. whether the user is a beginner or an expert), word-based data, cryptocurrency, virtual goods, and/or data collected in response to a survey (e.g. survey data submitted by the user or by a third-party, and optionally containing feedback data). This additional data can also be transmitted to the processing system 104 (step 214), for example by manually entering the additional data into the processing system 104, or automatically transmitting the data from another source (e.g. a weather data database).

At step 216, the processing system 104 can generate a digital asset based on the data. As used herein, the phrase “based on the data” can indicate that the digital asset is generated based on the data alone, or is generated based on the data combined with the secondary data, and/or the additional data, and/or other information (e.g. a non-activity related data, such as a background image or art). In some examples, as mentioned above, the smartphone 112 can carry an app for generating the digital asset based on the data. In one particular example, the app can be configured to apply a set of instructions to the data (and optionally to the secondary data and/or additional data and/or other information) to generate a digital image that includes a visual representation of the data, such as an abstract visual representation of the data or a figurative visual representation of the data, or a combination thereof. Alternatively the digital asset can be an audio representation of the data, such as music created based on the data. For example, the processing system can autonomously generate the digital asset (e.g. the asset can be generative art), or can generate the art using input from the user or a third party. The app can further display the digital image, e.g. in reduced-size (e.g. thumbnail) form or in true-size form, or can play the audio. In alternative examples, the data (and optionally the secondary data and/or additional data and/or other information) can be uploaded to an internet-based platform (e.g. a third-party platform) that can generate a digital asset based on the data, or imported into a software program that is installed on a laptop or desktop computer and that can generate a digital asset based on the data.

The digital asset can optionally be based on the data, or an analysis of the data. For example, the digital asset can be generated by applying set of instructions to raw force data collected by the force sensors, or by analyzing the raw force data to determine gait metrics and then applying a set of instructions to the gait metrics. For further example, the representation can be based on position data collected by a GPS sensor, or based on speed metrics determined by the position data.

The digital asset can optionally be generated proportionally to the data, for example by applying a multiplier to the data.

The digital asset can optionally be based on the data from a single activity (e.g. a single run), or on the data from several activities (e.g. a run and a bike ride, or an athletic activity and a gaming activity). The digital asset can optionally be based on the data from a single user, or on the data from multiple users (e.g. a teammate or opponent). Optionally, a cryptographically secured digital asset can be generated for multiple users. For example, each member of a winning team can receive a cryptographically secured digital asset.

The digital asset can optionally be created and/or cryptographically secured during the activity, upon completion of the activity or several activities, daily, weekly, or on another time basis.

The digital asset can optionally be automatically created in given circumstances. For example, in a race with multiple participants, the digital asset can automatically be created for the winner, or the top three finishers.

Optionally, several digital assets can be combined to create a combined digital asset. For example, a digital asset can be created for a runner, for each run completed. When the runner completes 100 runs, the digital assets can be combined (e.g. by applying a set of instructions to the digital assets) to create a digital asset that is based on the combined digital assets. For further example, a digital asset can be created for each runner in a race, and the digital assets can be combined (e.g. by applying a set of instructions to the digital assets) to create a digital asset that is based on the combined digital assets.

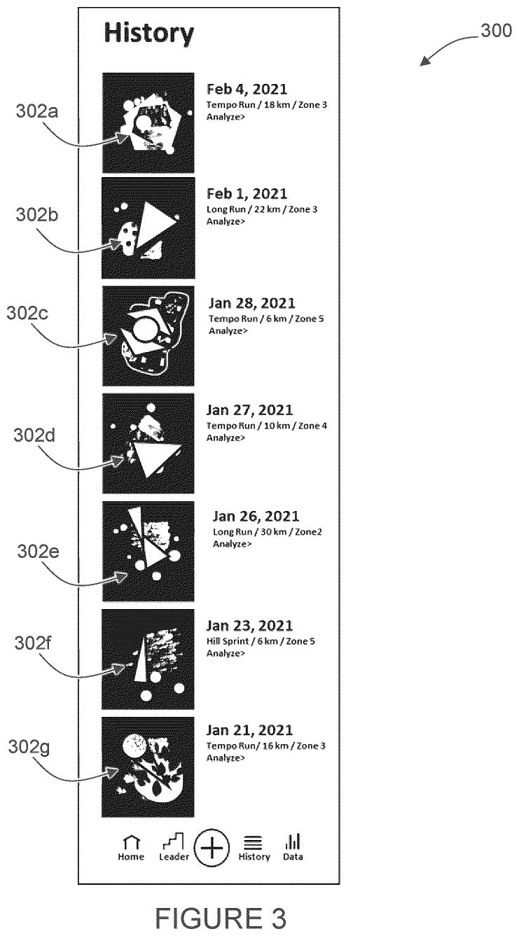

FIG. 3 shows an example user interface 300 for an app that can be used to generate a digital asset based on the data collected by activity data sensors 102, and to display a thumbnail of the digital asset. More specifically, FIG. 3 shows a user's activity history. A digital asset has been generated for each activity, by applying a set of instructions to the data collected during the activity, and a thumbnail 302a-302g of each digital asset is displayed in the user interface 300. In the example shown, the digital assets are in the form of digital images that include an abstract visual representation of the data. In the example shown, the digital asset is in the form of a two-dimensional abstract visual representation; however, in other examples, the visual representation may be one- or multi-dimensional. In further alternative examples, the digital asset may be of another form, such as an audio file. The thumbnail 302a-302g of each digital asset is displayed together with metadata relating to the activity, including date, the type of activity, and the distance covered during the activity. The metadata can be generated, for example, based on the secondary activity data (e.g. based data collected by a GPS sensor). While FIG. 3 is a black and white image, the digital assets may be color images, and the colors can optionally be selected by the algorithm.

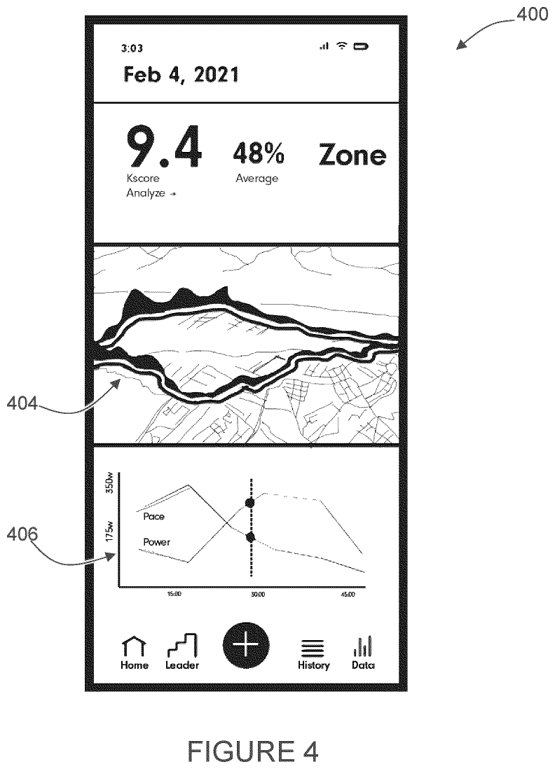

FIG. 4 shows another example user interface 400 for an app that can be used to generate a digital asset based on the data collected by activity data sensors 102, and display the digital asset and/or a thumbnail of the digital asset. In FIG. 4, the digital asset includes two portions 404, 406, each of which is generated based on the data and the secondary data. That is, the first portion 404 of the digital asset includes a figurative representation of the secondary data, in the form of a map that shows a user's route during an activity. The first portion 404 further shows a representation of the data collected by the activity data sensors 102 in the insoles 106, in the form of a graph superimposed on the map. The second portion 406 of the digital asset includes a visual representation of the data and the secondary data in the form of another graph.

Optionally, the method can include automatically detecting the activity that is being performed by the user. For example, based on the force data, the processing system 104 can determine whether the user is running or cycling.

Referring back to FIG. 2, at step 218, the digital asset can be cryptographically secured. Cryptographic securing can be carried out in a variety of ways. In some examples, the app that is used to generate the digital asset can also be used to cryptographically secure the digital asset. In some examples (e.g. as shown in FIG. 1), the app can be in communication with a blockchain enabled platform for cryptographically securing digital assets using blockchain technology. Such a platform can be, for example, a third-party platform (such as those mentioned above) or a platform that is purpose built for cryptographically securing the digital assets described herein.

The digital asset can optionally be cryptographically secured in given circumstances, such as when a milestone is reached. For example, in a race with multiple participants, the digital asset can automatically be cryptographically secured for the winner, or the top three finishers. For further example, in a gaming scenario, the digital asset can be automatically cryptographically secured when a certain milestone is reached in the gaming scenario, such as the completion of a battle, or landing on a leaderboard. In such instances, the digital asset can be generated based on the activity data generated in the reaching of the milestone.

Optionally, the generation of the digital asset and/or the cryptographic securing of the digital asset can depend on the difficulty of the activity. For example, based on the activity data and/or additional data, the processor can determine whether the activity was particularly difficult. For example, based on weather data, the processor can determine whether wind made an athletic activity particularly difficult, or based on activity data, the processor can determine whether an athletic activity was carried out fora particularly long duration. A unique or specific digital asset may be generated and/or cryptographically secured if the processor determines that the activity was particularly difficult. Alternatively, a digital asset may be cryptographically secured only if the processor determines that the activity was particularly difficult.

Ownership of the cryptographically secured digital asset can then optionally be transferred to a third party. For example the cryptographically secured digital asset can be gifted, donated, shared, traded, or sold, optionally using a purpose built platform or a third-party platform (such as those mentioned above). Optionally, the winner of a race or event can automatically be given ownership rights to the cryptographically secured digital asset. In some cases, the cryptographically secured digital asset can become a collector's item (e.g. in the case of an elite athletic performance). Optionally, the cryptographically secured digital asset can provide the owner with community rights, other ownership rights, or governance rights.

Optionally the cryptographically secured digital asset can provide the owner with the ability to unlock additional digital assets (e.g. the cryptographically secured digital asset can allow the owner to unlock a level in a video game).

In addition, the cryptographically secured digital asset can optionally be incorporated into a video game or virtual reality experience. For example, using the system and methods described above, a cryptographically secured digital asset in the form of a skin can be generated, based on data collected during an athletic and/or gaming activity. The skin can then be incorporated into a video game or virtual reality experience. For further example, using the system and methods describe above, an elite basketball player can generate a cryptographically secured digital asset based on data collected by the activity data sensors 102 during a particularly difficult or unique slam dunk. A layperson may then purchase or otherwise obtain that cryptographically secured digital asset, and can upload it into a gaming scenario, to execute the exact same slam dunk in a gaming scenario. In another example, a user can generate a cryptographically secured digital asset based on an activity, where the cryptographically secured digital asset is a representation of the user's gait. The user can then upload the cryptographically secured digital asset into a game or virtual reality experience, so that their gait is mimicked in the game or virtual reality experience. Alternatively, the user can acquire a cryptographically secured digital asset that represents another person's gait (e.g. a celebrity), and then upload the cryptographically secured digital asset into a game or virtual reality experience, so that the user can mimic the gait of the third party in the game or virtual reality experience. Alternatively, the cryptographically secured digital asset can be used to unlock features of a video game.

Optionally, prior to an event (e.g. a sporting event), the rights to a cryptographically secured digital asset that is to-be-created based on the event can be prepurchased.

Optionally, the digital asset can be converted to a representation on apparel.

The user can optionally be given revenue rights, or such rights can be allocated to coaches, other athletes, therapists, trainers, and/or others who have invested in the user.

Optionally, the user can be authenticated using the data. This may be particularly beneficial in the case of well-known users (e.g. celebrities), such as high-performance or professional athletes. Authentication can be based, for example, on gait metrics (e.g. a pressure sensing signature), or a motion signature (e.g. gait outliers).

Optionally, various other information, such as statistics relating to an activity (e.g. an athletic activity), can be authenticated using the data. For example, in the case of a running race, the data can be used to authenticate the results of the race (e.g. the winner of the race), or to authenticate a record (e.g. a personal best). For further example, the data can be used to authenticate whether a standard has been met (e.g. whether a run qualified the runner for an elite race such as the Boston marathon).

Optionally, a physical object can be created based on the digital asset. For example, a 3D-printer can print a physical representation of the digital asset (e.g. a sculpture).

Optionally, the systems described herein can be configured to generate different cryptographically secured digital assets with time. For example, in a gaming scenario, a certain cryptographically secured digital asset may be generated in a given season. Then, in a subsequent season, a different type of cryptographically secured digital asset may be generated. For further example, a cryptographically secured digital asset in the form of a certain skin may be available in the fall of a given year, or within a 24-hour period, or during a given week, or during a scheduled event, and may no longer be available after that time.

Optionally, cryptographically secured digital assets can be generated in order to encourage use of the systems described herein. For example, a user can earn a cryptographically secured digital asset after completing a certain number of activities. In a more specific example, in a gaming scenario, a user can earn a cryptographically secured digital asset in the form of a skin after completing a certain number of games or levels in the game. In another example, a user can earn a cryptographically secured digital asset when a particular time milestone is reached, such as one year of use of the system.

Optionally, the systems described herein can be configured to limit the number of cryptographically secured digital assets that can be generated in a given time frame. Optionally, the systems described herein can be configured to generate and/or cryptographically secure digital assets at certain time points (e.g. once a day, once a month, or once a year).

Optionally, a cryptographically secured digital asset may be generated and given to a user in exchange for the user sharing the activity data and or providing additional information. For example, a manufacturer of a sensor may benefit from receiving the activity data collected with an activity data sensor, as well as additional information (e.g. survey data relating to the activity). Accordingly, the manufacturer of the sensor may provide the user with a cryptographically secured digital asset in exchange for the activity sensor data and/or additional data. For further example, a user may be rewarded with a cryptographically secured digital asset in exchange for calibrating their sensor(s). That is, certain sensors may require calibration prior to use. This may particularly be the case for force sensors within insoles. With such insoles, a user may be instructed to stand in certain positions and/or perform certain movements, and then algorithms may be run to calibrate the sensors based on the data collected during the calibration. A digital asset may be generated based on the data collected during calibration, and the digital asset may be cryptographically secured and gifted to the user.

Optionally, the generation of the digital asset and/or the cryptographic securing of the digital asset can depend on the user's skill level. For example, for a user designated as a beginner, a digital asset may be generated and/or cryptographically secured where the activity data indicates that the user was consistent in the completion of the activity. For further example, for a user designated as an expert, a digital asset may be generated and/or cryptographically secured where the activity data indicates that the user has completed a near-impossible feat.

Optionally, the digital asset may initially be cryptographically secured as a fungible asset. Then, at a later time, the fungible asset may be further cryptographically secured as a non-fungible asset. For example, a fungible asset in the form of a skin may be further cryptographically secured as a non-fungible asset when the skin is used in a gaming scenario (e.g. when the user ‘wears’ the skin in a game, or when the skin is imported into a given platform).

Optionally, a combination of assets can be generated. For example, in the methods described herein, a first, a second, and a third cryptographically secured digital asset can be generated, and a real world prize can be generated (e.g. a trophy, entry into a sweepstakes, or registration in an event). The cryptographically secured digital assets may be fungible, non-fungible, or a combination thereof (e.g. the first and second cryptographically secured digital assets may be non-fungible, and the third cryptographically secured digital asset may be fungible).

Optionally, where the cryptographically secured digital asset is to be used in a gaming scenario (e.g. as a skin), the cryptographically secured digital asset can include a set of characteristics or descriptive attributes that can affect how the cryptographically secured digital asset is used in game play. For example a skin in the form of a jet-pack can be generated and cryptographically secured. The jetpack can have 60% power, 40% thrust, 75% speed, and 15% handling. Alternatively, the jetpack can have 30% power, 20% thrust, 30% speed, and 100% handling. Alternatively, the jetpack can have infinite fuel. The jetpack can optionally be a special jetpack that can, for example, be connected to a bicycle in a video game, to make a rocket bike. These attributes can optionally be based on the activity data, or can be random. In either case, the attributes may not be revealed to the user until the digital asset is generated and/or cryptographically secured (thus, there may be an “unboxing” experience for the user). Optionally, some such jetpacks may be fungible—e.g. there may be 1000 available jetpacks that are particularly fast, and 1000 available jetpacks that are particularly powerful. Further, some such jetpacks may be non-fungible—e.g. there may be only a single special jetpack.

Optionally, there may be a ceiling on the attributes associated with the cryptographically secured digital asset, or on the activity (e.g. the duration of the activity) based on which the cryptographically secured digital asset is generated.

In various alternative examples, particularly in a gaming scenario, the methods can include generating a digital asset based on only non-sensor based data (e.g. videographic data collected by a camera that is part of a gaming system) and cryptographically securing the digital asset.

While the above description provides examples of one or more processes or apparatuses or compositions, it will be appreciated that other processes or apparatuses or compositions may be within the scope of the accompanying claims.

To the extent any amendments, characterizations, or other assertions previously made (in this or in any related patent applications or patents, including any parent, sibling, or child) with respect to any art, prior or otherwise, could be construed as a disclaimer of any subject matter supported by the present disclosure of this application, Applicant hereby rescinds and retracts such disclaimer. Applicant also respectfully submits that any prior art previously considered in any related patent applications or patents, including any parent, sibling, or child, may need to be re-visited.

1. A method for creating a cryptographically secured digital asset, comprising:
a. using at least a first activity data sensor to collect data relating to an activity carried out by a user;
b. transmitting the data to a processing system;
c. using the processing system to generate a digital asset based on the data; and
d. cryptographically securing the digital asset.

2. The method of claim 1, wherein the first activity data sensor is a somatic sensor, and the somatic sensor is a force sensor, a shear sensor, a weight sensor, a body-mass-index sensor, a temperature sensor, a moisture sensors, a heart rate sensor, a heart rate variability sensor, a blood pressure sensor, a blood flow sensor, a cardiac output sensor, a perfusion sensor, an inductance sensor, a resistance sensor, a dielectric sensor, a capacitance sensor, a conductance sensor, an odor sensor, a taste sensor, a hydration sensor, a respiratory flow rate sensor, an accelerometer, a gyroscope, a magnetometer, a limb positioning measurement device, a sensor for detecting the presence of a chemical marker, a blood glucose sensor, a sweat glucose sensor, a blood alcohol sensor, an oxygen sensor, a carbon dioxide sensor, a drug level sensor, an electrolyte sensor, a pH sensor, an acidity sensor, an EEG sensor, an EMG sensor, an ECG sensor, a lung function meter, an impairment sensor, a sleep sensor, a body fat sensor, a height sensor, a fatigue sensor, an audio sensor, an RFID tag sensor, radar, lidar, an airflow meter, a spirometer, a lung volume sensor, a facial recognition sensor, a voice sensor, an emotion sensor, a stress sensor, an endorphin sensor, a cortisol sensor, an adrenaline sensor, an infrared sensor, or a bacterial load sensor.

3-4. (canceled)

5. The method of claim 1, wherein the first activity data sensor is a non-somatic sensor, and the non-somatic sensor is a timer, weather sensor, a GPS sensor, an audio sensor, a light sensor, an altimeter, radar, lidar, a milestone sensor, an equipment-based sensor, or a proximity sensor.

6. (canceled)

7. The method of claim 1, wherein step a. comprises collecting the data while the first activity data sensor is carried by a wearable device worn by the user, and the wearable device is an insole worn by the user.

8. (canceled)

9. The method of claim 1, wherein the activity comprises a gaming activity or athletic activity.

10-12. (canceled)

13. The method of claim 1, wherein the digital asset comprises a visual representation of the data.

14-19. (canceled)

20. The method of claim 1, further comprising using the data to authenticate the user, wherein:
the first activity data sensor is a force sensor positioned on a sole of the user's foot;
the data comprises force applied between the user's foot and a surface; and
using the data to authenticate the user comprises using the processing system to analyze the user's gait based on the data, and authenticating the user based on the analysis of the user's gait.

21-24. (canceled)

25. The method of claim 1, wherein the cryptographically secured digital asset comprises a skin or an attribute that is usable in gaming.

26-28. (canceled)

29. A system for creating a cryptographically secured digital asset, comprising:
at least a first activity data sensor configured to collect data relating to an activity carried out by a user; and
a processing system configured to receive the data from the first activity data sensor, generate a digital asset based on the data, and cryptographically secure the digital asset.

30. The system of claim 29, wherein the first activity data sensor is a somatic sensor, and the somatic sensor is a force sensor, a shear sensor, a weight sensor, a body-mass-index sensor, a temperature sensor, a moisture sensors, a heart rate sensor, a heart rate variability sensor, a blood pressure sensor, a blood flow sensor, a cardiac output sensor, a perfusion sensor, an inductance sensor, a resistance sensor, a dielectric sensor, a capacitance sensor, a conductance sensor, an odor sensor, a taste sensor, a hydration sensor, a respiratory flow rate sensor, an accelerometer, a gyroscope, a magnetometer, a limb positioning measurement device, a sensor for detecting the presence of a chemical marker, a blood glucose sensor, a sweat glucose sensor, a blood alcohol sensor, an oxygen sensor, a carbon dioxide sensor, a drug level sensor, an electrolyte sensor, a pH sensor, an acidity sensor, an EEG sensor, an EMG sensor, an ECG sensor, a lung function meter, an impairment sensor, a sleep sensor, a body fat sensor, a height sensor, a fatigue sensor, an audio sensor, an RFID tag sensor, radar, lidar, an airflow meter, a spirometer, a lung volume sensor, a facial recognition sensor, a voice sensor, an emotion sensor, a stress sensor, an endorphin sensor, a cortisol sensor, an adrenaline sensor, an infrared sensor, or a bacterial load sensor.

31-32. (canceled)

33. The system of claim 29, wherein first activity data sensor is a non-somatic sensor, and the non-somatic sensor is a timer, weather sensor, a GPS sensor, an audio sensor, a light sensor, an altimeter, a finish-line sensor, an equipment-based sensor, or a proximity sensor.

34. (canceled)

35. The system of claim 29, further comprising a wearable device, wherein the wearable device is an insole, or a piece of gaming equipment that carries the first activity data sensor.

36-37. (canceled)

38. The system of claim 29, wherein the digital asset comprises a visual representation of the data.

39. The system of claim 29, wherein the processing system is configured to apply a set of instructions to the data to generate the digital asset.

40-42. (canceled)

43. The system of claim 29, wherein the processing system is configured to generate the digital asset based on the data and additional non-sensor based data relating to the activity carried out by the user.

44. (canceled)

45. The system of claim 29, wherein the processing system is configured to use the data to authenticate the user, wherein:
the first activity data sensor is a force sensor that is positionable on a sole of the user's foot and that is configured to measure force applied between the user's foot and a surface; and
the processing device is configured to analyze the user's gait based on the data, and authenticate the user based on the analysis of the user's gait.

46. (canceled)

47. The system of claim 29, wherein the processing system is configured to use the data to authenticate at least one statistic relating to the activity.

48. The system of claim 29, wherein the processing system is configured to automatically detect a nature of the activity.

49. (canceled)

50. The system of claim 29, wherein the system is configured to generate the digital asset proportionally to the data.

51. The method of claim 29, wherein the cryptographically secured digital asset comprises an attribute or a skin that is usable in gaming.

52-70. (canceled)
